(19) United States
(12) Patent Application Publication
(10) Pub. No.: US 2024/0175821 A1
(43) Pub. Date: May 30, 2024

(54) INTELLIGENT READER AND DATA RECORDING SYSTEM FOR BIOLOGICAL, CHEMICAL, AND INSTANT BIOLOGICAL AND ENZYMATIC STERILIZATION INDICATORS

(71) Applicant: Thomas James Riha, Perrysburg, OH (US)

(72) Inventor: Thomas James Riha, Perrysburg, OH (US)

(21) Appl. No.: 18/435,587

(22) Filed: Feb. 7, 2024

Related U.S. Application Data
(63) Continuation of application No. 17/302,606, filed on May 7, 2021.
(60) Provisional application No. 63/021,538, filed on May 7, 2020.

(51) Int. Cl. G01N 21/78; A61L 2/28; G01N 21/25; G16H 40/40

(52) U.S. Cl. CPC G01N 21/78; A61L 2/28; G01N 21/25; G16H 40/40

(57) ABSTRACT

A system for measuring a sufficiency of a sterilization process includes a vessel associated with a specimen exposed during the sterilization process, wherein a scannable code is directly connected to the vessel, and wherein the scannable code is adapted to provide sterilization data associated with the sterilization process. Additionally, a biological indicator is physically associated with the vessel, and a computing device is configured to receive the sterilization data so that the computing device renders a pass/fail determination of the sufficiency of the sterilization process through comparing a color of the biological indicator against a color of a reference standard defined by the sterilization data.

### CROSS-REFERENCE TO RELATED APPLICATION

This application claims the benefit of priority of U.S. Non-Provisional application Ser. No. 17/302,606, filed May 7, 2021, as a Continuation thereof, and thus claim benefit, therethrough, of U.S. Provisional Application No. 63/021,538, filed May 7, 2020, the contents of both are herein incorporated by reference.

### BACKGROUND OF THE INVENTION

The present invention relates to sterilization data reading, recording and logging devices, and more particularly, to an intelligent reader and data recording system for biological, chemical, instant biological and enzymatic sterilization indicators, and protein sensor indicators exogenous or endogenous to a bacterial spore, and a method for its use.

Biological and Chemical indicators are used throughout the healthcare industry to monitor and determine the efficacy of Steam and Vaporized Hydrogen Peroxide sterilization. Currently available systems and processes are slow and limited. Further results are not capable of being logged by currently available systems.

Results of biological indicators used for these processes currently take 20 minutes to over 1 hour to determine the results. Current system equipment that determines these results is capable only of logging a biological indicator result by heating the biological indicator (to a temperature of between 55° C.-65° C.), and then utilizing UV spectroscopy, with detection of enzymes with substrates that fluoresce to indicate viable (alive) bacterial spores. Chemical Indicators and any result that is visually produced (Instant IBI or traditional biological indicators) are not able to be logged by the current systems readily used in the healthcare industry.

Other devices currently available in the market do not determine and log the results of a chemical indicator and biological or enzymatic indicator or protein indicator, or any visual result all at once.

Further, currently available systems and devices are not portable. Current systems consist of stationary pieces of equipment which occupy permanent counter space in the location where they are used.

A need exists for an intelligent reader and data recording system for biological, chemical, instant biological, enzymatic and or protein sensor based sterilization indicators which provides instant results for which data may be easily captured, logged and stored. A further need exists for such a system which is fast, efficient, and versatile, in addition to being portable and capable of being stored while not in use.

### SUMMARY OF THE INVENTION

Advantageously, the present invention provides an unprecedented solution to the aforementioned problems. In one aspect, the present invention provides an intelligent reader and data recording system for biological, chemical, and instant biological and enzymatic sterilization indicators, and protein sensor indicators. The system of the present invention provides instant results for which data may be easily captured, logged and stored.

In another aspect, the present invention provides an intelligent reader and data recording system for biological, chemical, and instant biological and enzymatic sterilization indicators and protein sensors exogenous or endogenous to a bacterial spore, the system comprising a camera, and system software.

In a further aspect, the intelligent reader and data recording system for sterilization indicators may comprise a server, software, an electronic device having an integral camera, and an aperture box having at least one UV filter lens, that is removably attachable to the camera.

In a still further aspect the present invention provides a data recording method comprising providing a system as described herein, and using the system to read and record sterilization data from a testing product. The invention software, installed on a cell phone, may use the cell phone camera to scan the barcode or QR code of the testing product. A first scanned image of the contents of the testing product may be taken to receive a pass or fail determination based on the system software's pre-loaded reference standards for color of chemical indicators or enzymatic indicators. The instantaneous pass or fail results are generated and uploaded to desired server. If biological detection is needed, an aperture box having UV filtration lenses may be removably attached in front of the cell phone camera. The product may then be placed directly in front of the aperture box lenses. Then a second scan of the product may be taken, using the camera and the aperture box UV filtration lenses, to detect biological or enzymatic indicators. Instantaneous pass or fail results may be generated and uploaded to the system server.

In another aspect, the present invention provides a system and method as described which is fast, efficient, and versatile. The system of the present invention provides instant results for which data may be easily captured, logged and stored. In a further aspect, the system of the present invention is portable and capable of being stored while not in use, and does not take up any permanent counter space.

These and other features, aspects and advantages of the present invention will become better understood with reference to the following drawings, description and claims.

### BRIEF DESCRIPTION OF THE DRAWINGS

The preferred embodiment of the claimed subject matter will hereinafter be described in conjunction with the appended drawings provided to illustrate and not to limit the scope of the claimed subject matter, where like designations denote like elements, and in which:

FIG. 1 is a schematic side elevation view of the invention in use;

FIG. 2 is an exploded perspective view of the invention;

FIG. 3 is a perspective view of the invention;

FIG. 4 is a front elevation view of the invention;

FIG. 5 is a partial cross sectional view along line 5-5 of FIG. 4, providing a side elevation view enlarged to show detail; and

FIG. 6 is the flowchart of the invention.

It is to be understood that like reference numerals refer to like parts throughout the several views of the drawings.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

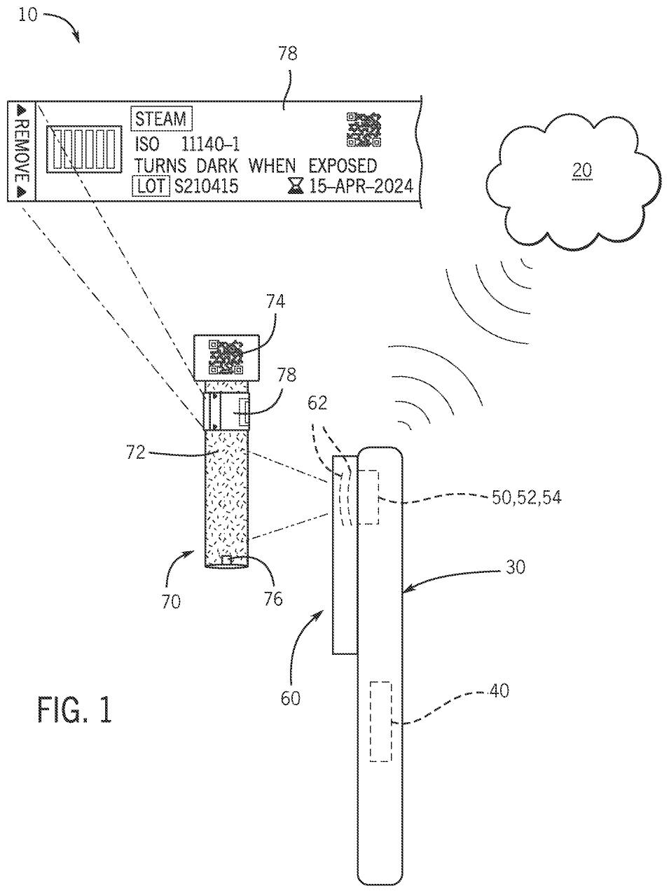

As used herein, “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, as there is no intention to be bound by any express or implied theory presented in the preceding technical field, background, brief summary or the following detailed description, it is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Broadly, one embodiment of the present invention provides an intelligent reader and data recording system for biological, chemical, and instant biological and enzymatic sterilization indicators and protein sensors, and a method for its use.

The system of the present invention solves the problems associated with reading and recording data for biological, chemical, and instant biological and enzymatic sterilization indicators and protein sensors.

As noted hereinabove, biological and chemical indicators are used throughout the healthcare industry to monitor and determine the efficacy of Steam and Vaporized Hydrogen Peroxide sterilization. Currently, existing processes take from about 20 minutes to over 1 hour to determine the results. Currently available equipment that determines these results is capable only of logging the biological indicator result by heating the biological indicator to a temperature of between 55° C.-65° C.; and utilizing UV spectroscopy with detection of enzymes by the use of substrates that fluoresce to indicate the presence of viable (living) bacterial spores. Current options determine results with specific incubator equipment which relies on fluorescent detection of an enzyme alpha-glucosidase with a fluorescent substrate which may be but is not limited to, MUG.

The claimed invention differs from and distinguishes over what currently exists. There are no products that are able to determine pass or fail of all three types of sterilization monitoring products, chemical, biological and enzymatic or protein indicators, and then log the result without the need of a rapid incubator with UV detection.

The present invention provides an unprecedented solution There is no current system that is able to instantly detect and determine pass or fail results of biological, chemical, and enzymatic indicators by means of a camera and custom software, one or more of which may be provided on an electronic device such as a cell phone, as the present invention provides. The present invention may further be used in association with protein sensors.

Moreover, currently available systems that are used in the health care industry are not able to log chemical Indicators, or any result that is visually produced (such as traditional biological indicators or Instant Biological Indicators (IBI)). Currently available options do not determine and log the results of a chemical indicator and biological, enzymatic or protein sensor indicator exogenous or endogenous to a bacterial spore and detected by UV fluorescence, or any visual result all at once.

Current systems require the use of one particular product to be used in the system from specific manufacturers and none of the current systems can log and determine chemical indicator results. These current systems take up to 20 minutes or longer to determine and log the results for biological and enzymatic results and protein sensors.

In addition, current systems include stationary equipment provided as a permanent installation—these systems are not portable, and take up counter space.

The advantages of the system of the present invention are many. The system of the present invention provides an unprecedented solution to all of these problems associated with previous systems. The present invention provides an intelligent reader and data recording system for biological, chemical, and instant biological and enzymatic or protein sterilization indicators and protein sensors or indicators for any uses. The system of the present invention is user-friendly, fast, efficient, and versatile, and is capable of use to provide instant results for which data may be easily captured, logged and stored. Further, the system of the present invention is portable and capable of being easily stored while not in use, and does not take up any permanent counter space.

In an exemplary embodiment, the intelligent reader and data recording system for sterilization indicators may comprise a camera, and system software. The intelligent reader and data recording system for sterilization indicators may comprise a server, an electronic device having an integral camera including an aperture box with UV filter lenses, and software.

The system of the present invention is capable of determining and logging results of chemical, biological, and enzymatic or protein sensor indicators used for sterilization and disinfection with little end user input; and instantly determining the results and logging the results within a data tracking system of choice, all on a portable electronic device with a camera. The system of the present invention may comprise an instant detection system capable of use with any suitable manufacturer's testing product. The system of the present invention may be used with all testing products including the Instant IBI product manufactured by True Indicating.

More particularly, the system of the present invention is capable of use to identify and determine pass or fail results and digitally log the result for biological, chemical, or enzymatic indicators used to monitor sterilization and disinfection processes. The system may include and utilize a smartphone camera, tablet camera, or a standalone camera, and custom system software which references standards for pass or fail by results of one or more of colorimetry or fluorescence. The camera may be placed in view of the biological, chemical, enzymatic or protein indicator pre or post-sterilization cycle, and the image seen by the camera may be uploaded to a data tracking system. The result may be compared to a reference standard which signifies a pass or fail result, and then reports to the end user that the sterilization or disinfection cycle passed or failed based on the result detected by the camera and software.

The system may include at least one computer with a user interface. The computer may include at least one processing unit coupled to a form of memory. The computer may include, but not limited to, a microprocessor, a server, a desktop, laptop, and smart device, such as, a tablet and smartphone. The computer includes a program product including a machine-readable program code for causing, when executed, the computer to perform steps. The program product may include software which may either be loaded onto the computer or accessed by the computer. The loaded software may include an application on a smart device. The software may be accessed by the computer using a web browser. The computer may access the software via the web browser using the internet, extranet, intranet, host server, internet cloud and the like.

The ordered combination of various ad hoc and automated tasks in the presently disclosed platform necessarily achieve technological improvements through the specific processes described more in detail below. In addition, the unconventional and unique aspects of these specific automation processes represent a sharp contrast to merely providing a well-known or routine environment for performing a manual or mental task.

Referring to FIGS. 1-5, the intelligent reader and data recording system for biological, chemical and instant biological and enzymatic sterilization indicators, is shown generally at 10.

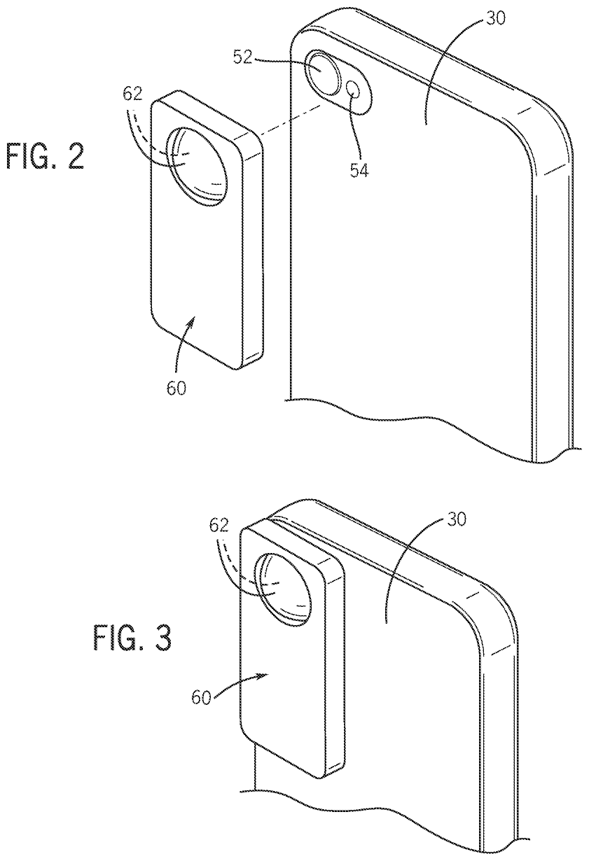

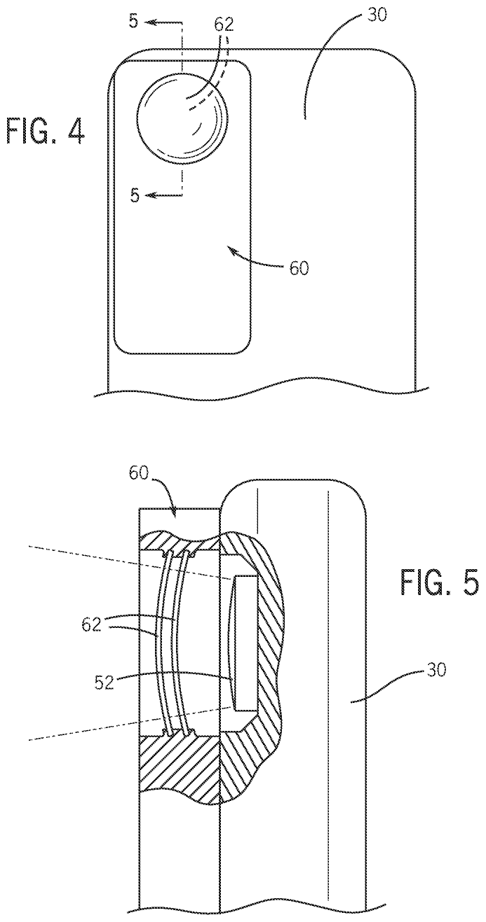

FIG. 1 provides a schematic side elevation view of the system of the present invention in use. FIG. 2 provides an exploded perspective view of the invention. FIG. 3 is a perspective view of the invention. FIG. 4 provides a front elevation view of the invention and FIG. 5 is a partial side elevation view taken along line 5-5 of FIG. 4, with parts broken away and enlarged to show detail.

The system 10 may include a server 20, an electronic device 30 which includes a software application 40 loaded thereon, and a camera 50 having a LED light source 52 and a camera lens 54. The system may further include an aperture box 60 for placement over the camera lens 52. The aperture box 60 may include one or more UV filter lenses 62.

The system 10 may be used with a sterilization testing product 70 having a sample 72, associated with an identifier such as a QR code 74 and an indicator 76 and a chemical indicator CI strip 78.

The electronic device 30 may be any suitable electronic device such as, for example without limitation, a smartphone, tablet, computer. In the embodiment shown at FIG. 1, the electronic device 30 may be a mobile phone.

The software application 40 is a custom system software comprising Intelligent Sterilization Result Software Application which may be provided for Android, Apple IOS, and or web-based platforms.

The camera 50 may be at least one camera. The camera may be any suitable camera. In some embodiments of the system the camera may be integral to the electronic device. In other embodiments the camera may be a separate device. In further embodiments the system may include at least one camera selected from cameras integral to an electronic device, cameras which are separate from the device, and combinations thereof. The system may be capable of use with more than one camera as needed.

The camera 50 comprises an LED light 52 incorporated therein and a camera lens 54. The LED light 52 may be any suitable LED light, nonlimiting examples of which may be standard LED light or LED UV light, or any other light source capable of producing the necessary wavelengths for detection. In one embodiment the LED light may be a LED UV with wavelengths of between about 200-1000 nm.

The camera and software are necessary for determining the chemical, biological, enzymatic, and or protein sensor indicator results.

In some embodiments the system 10 further comprises a small aperture box 60 which may be removably placed over the lens 54 of the camera 50. The aperture box 60 may include at least one filter capable of use to filter the most favorable UV wavelengths to perform UV fluorescence detections. In some embodiments the aperture box 60 may include a plurality of filters.

The system and its components may be made of any suitable material, by any suitable fabrication process.

The aperture box may be made of any plastic and may include specific lenses to filter UV, and a UV LED source is included which is powered by a standard outlet, or a smartphone or tablet battery. In some embodiments, the aperture box may be optional, and may be including in embodiments assessing fluorescence results of a biological indicator.

The attachment means for removable attachment of the aperture box over the camera may be any suitable attachment device or method, nonlimiting examples of which may include mechanical attachment devices such as snap fit, press fit, hook and loop fasteners or clips, or any suitable adhesive tape.

In use, the camera 50 from any smartphone, tablet, or a traditional camera may be used to view the sterilization testing product 70 and sample 72. The sample is associated with an identifier such as a QR code 74 and an indicator 76 and a chemical indicator CI strip 78. The camera is able to “see” the color result of an indicator 76, such as, for example without limitation, a chemical or biological indicator, enzymatic, and or protein sensor indicator. If utilizing UV spectroscopy for a biological indicator, the camera 50 may detect fluorescence. The camera 50 may be operably connected to an aperture box 60 including one or more lenses 62 capable of filtering UV light to enhance the camera's function in detecting fluorescence. The camera 50 may also include an LED 52. The LED 52 may be a UV LED source necessary for fluorescence detection, if present on the model of camera being used. The system Intelligent Sterilization Result software application may be used to scan and determine the product 70 being used by QR or RFID (see QR code 74 at FIG. 1) and may compare the visual result to previously loaded reference standard for that specific product 70 located in the database 20. The reference standard may be any suitable reference standard, nonlimiting examples of which may include Hunter LAB color reference or similar, or UV wavelength reference, or similar. If the result is within the set range of pass, the software application 40 logs the result as pass. If the result is too different from the sterilization monitoring product reference, the result is reported as a fail. The aperture box 60 may be used when UV spectroscopy is utilized. The aperture box 60 may include one or more specific lenses 62 capable of use to filter out unwanted UV wavelengths in order to focus the most favorable UV wavelength between 200-400 nm for detection of a fluorescent result to detect pass or fail of the potential growth of Geobacillus stearothermophilus spores or a protein sensor exogenous or endogenous to the bacterial spores within a biological indicator. In some embodiments, the aperture box 60 may also supply the necessary UV LED source if the UV LED source is not present in the camera being used. The camera 50 in conjunction with the aperture box 60 may detect the fluorescent response produced by spores which are still alive within a biological indicator and react with a substrate within the biological indicator media. When a fluorescent result of the biological indicator 76 is detected, this signifies a positive result (fail) and spores are alive. When spores are not detected, no fluorescence response is detected and the biological result is considered negative (pass).

The camera lens 54 may visually see color of chemical indicators 78 or enzymatic indicators, or fluorescence results of biological, enzymatic or protein sensor indicators 76. In one embodiment the camera may be associated with a protein sensor. The camera 50 supplies the image(s) to the Intelligent Sterilization Result System Software 40. The software 40 uses the supplied image(s) from the camera 50 and determines the specific product 70 by means of QR code 74 or RFID technologies, then compares the visual result being processed by the camera 50 against a pre-loaded reference standard for the product and determines pass or fail results. The pass or fail results may be based on Hunter LAB color (spectrodensitometer) or UV spectroscopy (200-400 nm). If the result does not match the reference standard for the specific product, then the result is logged as FAIL. If the result does match the reference standard of the specific product, then the result is logged as PASS. If using fluorescence, the aperture box 60 may be used to filter UV wavelengths and focus the most favorable wavelength between 200-400 nm to detect the outgrowth of spores. The results are then locally saved, the results may be accessed by the user to add specific information related to the exposure (such as, for example without limitation: load, tech, date or time, or the like). The results may further be uploaded to a database of choice.

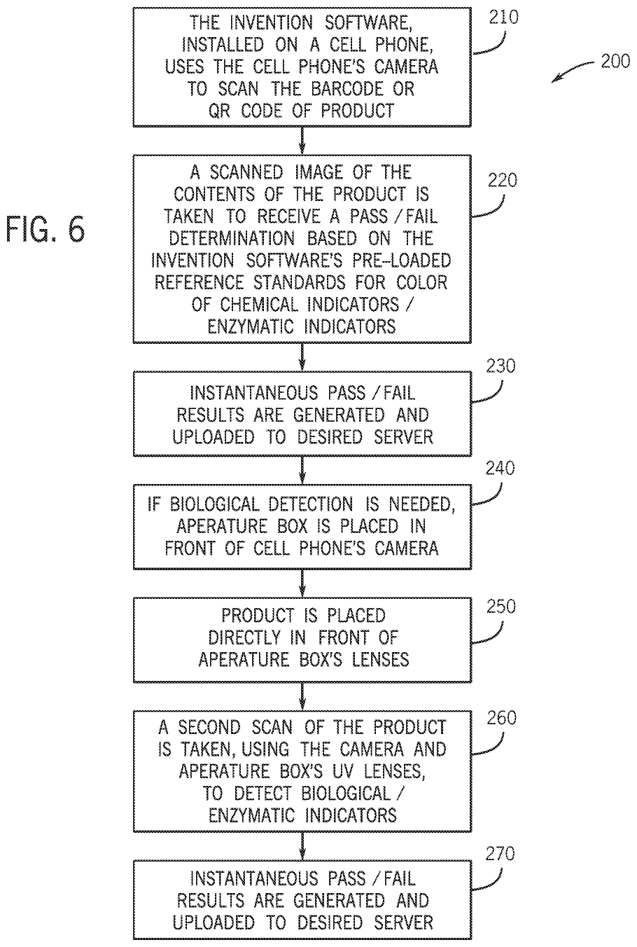

Referring now to FIG. 6, a flowchart of a method 200 of use of the system in accordance with the present invention is shown. The method of the present invention provides an intelligent reader and data recording method for reading and recording data of biological, chemical, and instant biological and enzymatic sterilization indicators. The data recording method 200 comprises providing a system as described herein, and using the system to read and record sterilization data and provides one or more of the following steps and combinations thereof. At Step 210, he invention software, installed on a cell phone, uses the cell phone's camera to scan the barcode or QR code of a product. At Step 220, scanned image of the contents of the product is taken to receive a pass or fail determination based on the invention software's pre-loaded reference standards for color of chemical indicators or enzymatic indicators. At Step 230, the instantaneous pass or fail results are generated and uploaded to desired server. At Step 240, if biological detection is needed, an aperture box is placed in front of cell phone's camera. At Step 250, the product is placed directly in front of aperture box's lenses. At Step 260, then a second scan of the product is taken, using the camera and aperture box's UV lenses, to detect biological or enzymatic indicators. At Step 270, instantaneous pass or fail results are generated and uploaded to the system server.

To make the system of the invention, one must build the software that is able to complete the requisite tasks and provide the user with the useful tool described above. The intelligent sterilization result software may be coded in either iOS or Android open source code, or web-based source application. References for a given product may be coded into the software for determining pass or fail results by means of Hunter LAB data, or UV wavelength (200-400 nm).

In an exemplary embodiment, the system of the present invention may be used to determine success of a sterilization process or cycle. A typical sterilization exposure for steam, ethylene oxide, hydrogen peroxide, steam formaldehyde, radiation or UV, etc. requires the use of a monitoring product which provides information on the success of the sterilization cycle. Currently, chemical indicators or any visual results are seen only by an end user who then determines whether a result passed or failed. The system of the present invention removes the need of an end user to determine any result of a sterilization monitoring product that is a chemical, biological or instant (enzymatic) result. An indicator selected from chemical indicator, biological indicators, enzymatic indicators, protein-based indicators or combinations thereof may be utilized to monitor the sterilization exposure. At the end of exposure, one may obtain the indicator used within the exposure, place onto a flat surface, and the monitoring product may be scanned with a smartphone camera, tablet camera, or discrete camera which has the inventive system software (inventive intelligent sterilization result software) loaded thereon. The software may capture the product information through use of the camera, and automatically determine which product is in view of the camera by means of a QR code, RFID or bar code. The software may compare the product to a loaded standard reference for a pass or fail result, and may immediately determine pass or fail of the product's result by means of color, fluorescence detection or both. This process may also be done for positive controls such as pre-exposure of product. If utilizing UV detection, an aperture box may be used in front of the camera lens to filter out unwanted UV wavelengths necessary for determining fluorescence from outgrowth of spores within a biological indicator. All results may be logged by the software and may provide the user the ability to type in fields such as, for example without limitation, Sterilizer ID, cycle number, load contents. The software may auto populate the time and date of the result report. The data may be saved in the user's desired location by means of Internet connection to one or more of a server address and email address.

In some embodiments the system may be used by a standalone camera with a standalone computer which has access to the system software, for intelligent sterilization result software.

Though the system of the present invention may include a software application capable of operating on any mobile device, in some embodiments the system may also work with a stationary device. The system may be operably connected to any camera capable of taking a picture of the instant indicator within the first 20 seconds of the indicator being exposed to a test specimen. The software application may further be able to scan, detect, and report pass or fail results post-sterilization for various indicators regardless of the sterilization processes.

In some embodiments, an indicator solution may be added to an established sample. Post addition of the indicator solution, the system and software application take a picture or scan of an indicator and compare the color of the product to an established sample color. The reference may be an established sample color or a pantone reference color for an indicator. The indicator may be chemical, biological, enzymatic or protein-based. If the developed color of the indicator is equal to or darker than the comparison reference sample, the system may designate the sterilization cycle as a failed cycle. If the color of the photo or scan is lighter than the established color, the system may designate the sterilization cycle as a passed cycle for the indicator. The system and application may also determine the pass or fail results of a chemical indicator based on the color of the chemical indicator post-exposure by matching, or not matching the established post-exposure reference color within the system.

The system is capable of identifying a sample by scanning a QR code or bar code. Whether the product passes or fails a testing event, in some embodiments, the system may also permit the user to enter relevant data for the sterilization cycle for the sample(s) scanned. In some embodiments the relevant data may include but would not be limited to the following:
1. Manufacturer of Equipment
2. Equipment #
3. Equipment Description
4. Pass or Fail of Product and cycle tested
5. Expiration Date of Product
6. Lot Number of Product
7. Date of test
8. Product Code
9. Temperature of sterilization process
10. Cycle time
11. User

In some embodiments, after a user enters relevant data, the system and software may automatically upload the indicator data (for indicators including but not limited to biological, chemical, enzyme and protein), using any traceability or tracking system, including any server designated by the user, including a server on the cloud supported by True Indicating for future access by individual users by unique account with unique login credentials.

The system may have alternative designs. In some embodiments, the system of the present invention may include a stationery device constructed and arranged to automatically apply an indicator added or applied by any device, such as, for example without limitation, a needle and syringe, which would allow for indicator solution comprising one or more of a biological, chemical, enzyme, protein, co-enzyme, other reactive substrate, protein sensor, and combinations thereof, to be applied to a biological tablet of the instant biological indicator. A camera would capture the color of the biological tablet within a specific time frame (timer built-in) and automatically determine pass and fail results based on developed colors as described above.

In some embodiments, the stationary device may also be used to incubate the biological spores and media for standard outgrowth of bacteria at necessary elevated temperatures. The stationary device may further be able to permit any indicator to be scanned and logged by the system as described previously.

In some embodiments the data recording system may comprise a stationary device having a first chamber disposing a camera, a second chamber disposing an indicator solution, and a computer including a data logging application; and a vessel disposing a biological material. A portion of the vessel may be inserted into the stationary device and disposed within the first chamber such that the camera scans the biological material for various chemicals and the data logging application analyzes the scan to determine the status of the biological material.

In some embodiments the status of a sterilization cycle may be determined based on the color of the product compared to an established sample color post-addition of the indicator solution for an enzymatic indicator. The data logging application may determine that the sterilization cycle has failed when the color is equal to or darker than the established sample color. The data logging application may determine that the sterilization cycle has passed when the biological material is lighter than the established sample color.

In some embodiments the data logging application automatically uploads the status of the biological material to a data logging server.

In some embodiments the stationary device may further comprise a third chamber disposing a needle; the third chamber in fluid communication with the first chamber and second chamber; and the needle is in fluid communication with the indicator solution. The needle may directly contact the vessel and inject indicator solution into the vessel such that the indicator solution interacts with the biological material. The stationary device may incubate the biological material when the vessel is disposed within the stationary device.

In summary, an intelligent reader and data recording system, method and software for sterilization indicators provides instant results for which data may be easily captured, automatically determined, logged and stored. The sterilization indicator may include biological, chemical, instant biological, enzymatic, protein sensor exogenous or endogenous to a bacterial spore, and combinations thereof. The system may include any computing device, a camera, system software, and a system server. The camera may be a standalone, or integral to the computing device, nonlimiting examples of which may be a cell phone or tablet. An aperture box including at least one UV filter lens may be removably installed over the camera lens. The system may be used to read and record sterilization data from a sterilization monitoring product. The software may be installed on the computing device camera or standalone camera, and use the camera to scan a barcode or QR code of the sterilization monitoring product. A first scanned image of the contents of the sterilization monitoring product may be taken to receive a pass or fail determination based on system software pre-loaded reference standards for color of chemical indicators or UV detection/colorimetry for biological enzymatic and or protein sensor based sterilization indicators. Instantaneous pass or fail results are generated and uploaded to the system server. If biological detection is needed, an aperture box having UV filtration lenses may be removably attached over the cell phone camera lens. The testing product may then be placed directly in front of the aperture box lenses. Then a second scan of the product may be taken, using the camera and the aperture box UV filtration lenses, to detect one or more of biological, enzymatic and protein sensor based indicators. Instantaneous pass or fail results may be generated and uploaded to the system server.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications, variations and changes in detail may be made without departing from the spirit and scope of the invention as set forth in the following claims.

What is claimed is:

1. A system for measuring a sufficiency of a sterilization process, the system comprising:
a vessel associated with a specimen exposed during the sterilization process;
a scannable code directly connected to the vessel, wherein the scannable code is configured to provide sterilization data associated with the sterilization process;
a biological indicator physically associated with the vessel;
a computing device configured to receive the sterilization data; and
the computing device configured to render a pass/fail determination of the sufficiency of the sterilization process through comparing a color of the biological indicator against a color of a reference standard defined by the sterilization data.

2. The system of claim 1, wherein the computing device further comprises a scanner configured to scan the scannable code for receiving the sterilization data.

3. The system of claim 1, wherein the computing device further comprises a camera configured to capture an image of the color of the biological indicator.

4. The system of claim 1, wherein the sterilization data comprise a date of the sterilization process.

5. The system of claim 1, wherein the sterilization data comprise a duration of the sterilization process.

6. The system of claim 1, wherein the sterilization data comprise a temperature of the sterilization process.

7. The system of claim 1, wherein the sterilization data comprise an expiration date of the specimen.

8. The system of claim 1, wherein the sterilization data comprise a lot number of the specimen.

9. The system of claim 1, further comprising a chemical indicator connected to the vessel wherein the chemical indicator is configured to indicate if the sterilization process happened.

10. The system of claim 9, wherein the chemical indicator is printed on a strip, wherein the strip is directly connected to the vessel.

11. The system of claim 1, wherein the comparison between said colors is based on spectrodensitometeric analysis.

12. The system of claim 1, further comprising a chemical indicator placed on a tray subject to the sterilization process, wherein the chemical indicator is configured to indicate if the sterilization process happened.

13. The system of claim 12, wherein the chemical indicator is printed on a strip, wherein the strip is directly placed on said tray.

14. The system of claim 12, wherein the computing device further comprises a camera configured to capture an image of the color of the chemical indicator.
